(19) United States
(12) Patent Application Publication
(10) Pub. No.: US 2009/0155768 A1
(43) Pub. Date: Jun. 18, 2009

(54) Reporter plasmid phage packaging system for detection of bacteria

(76) Inventors: Dean Scholl, Burlingame, CA (US); Carl R. Merril, Bethesda, MD (US); Sankar Adhya, Gaithersburg, MD (US); Michael McKinstry, Fairmont, WV (US); Rotem Edgar, Potomac, MD (US)

Correspondence Address: KNOBBE, MARTENS, OLSON & BEAR, LLP, 2040 MAIN STREET, FOURTEENTH FLOOR, IRVINE, CA, 92614, US

(21) Appl. No.: 11/547,587

(22) PCT Filed: Apr. 7, 2005
(86) PCT No.: PCT/US05/11607

§ 371 Date: Feb. 26, 2009

Related U.S. Application Data
(60) Provisional application No. 60/560,392, filed on Apr. 7, 2004.

(51) Int. Cl. C12Q 1/70; C12N 15/00; C12N 15/87

(52) U.S. Cl. 435/5; 435/320.1; 435/456

(57) ABSTRACT

The invention is related to a transducing particle that comprises a bacteriophage coat and a DNA core that comprises plasmid DNA comprising: a) a host-specific bacteriophage packaging site wherein the packaging site is substantially in isolation from sequences naturally occurring adjacent thereto in the bacteriophage genome, b) a reporter gene, c) a bacteria-specific promoter operably linked to said reporter gene, d) a bacteria-specific origin of replication, and optionally e) an antibiotic resistance gene. The invention includes phage transducing particles, methods of making transducing particles, and methods of using the transducing particles in bacterial detection.

### RELATED APPLICATIONS

This application claims the benefit of U.S. Provisional Application No. 60/560,392, filed Apr. 7, 2004, which is hereby incorporated by reference in its entirety.

### FIELD OF THE INVENTION

This invention is related to a reporter plasmid phage packaging system for detection of bacteria. It includes phage transducing particles, methods of making transducing particles, and methods of using the transducing particles in bacterial detection.

### BACKGROUND OF THE INVENTION

Bacteriophage are typically highly specific for a given species or strain of bacteria. This specificity can be exploited for the detection of a given species/strain of bacteria from an environmental or medical sample that may contain many different bacteria types. One strategy is to engineer reporter genes such as luciferase or green fluorescent protein into the phage genome such that the reporter gene is expressed and can be detected upon infection of the target bacteria. This concept is well documented. Nevertheless, this system has limitations, particularly when the only phages available for a given bacteria species are lytic and can rapidly kill the target cell before there is significant expression of the reporter gene.

### SUMMARY OF THE INVENTION

This new invention called “transducing particles” overcomes the above-mentioned limitations but still makes use of the specificity of bacteriophage to its host. Briefly, a plasmid which is capable of stable replication in a given host is engineered to contain a reporter gene as well as the phage packaging site (the specific DNA sequence on the phage genome that is required for genome packaging into the virion). This construct is transformed into a bacterial host, and then infected with the specific bacteriophage. Because the packaging site is on the plasmid, a percentage of the progeny phage particles will have reporter plasmids packaged into the heads instead of the phage genome. When these particles are contacted with target bacteria, the phage will inject the plasmid into the cell that can then be detected by expression of the reporter gene.

Commercial applications: detection of specific bacteria in an environmental or medical sample. Commercial uses: biodefense, food industry for contaminating bacteria, medical diagnosis for infectious disease. General method for packaging any DNA into a phage capsid, could be used for gene therapy etc.

### BRIEF DESCRIPTION OF THE DRAWINGS

FIG. 1. Detection of specific bacteria using luciferase reporter phage.

FIG. 2. Bacteriophage A1122.

FIG. 3. Bacteriophage T7 Genome and inserted GFP reporter gene.

FIG. 4. Reporter phage-induced fluorescence.

FIG. 5. Optimization of the reporter phage.

FIG. 6. Reporter plasmid packaging system.

FIG. 7. Additional studies.

FIG. 8. Transducing particles carrying plasmid expressing GFPuv.

FIG. 9. E. coli expressing GFPuv.

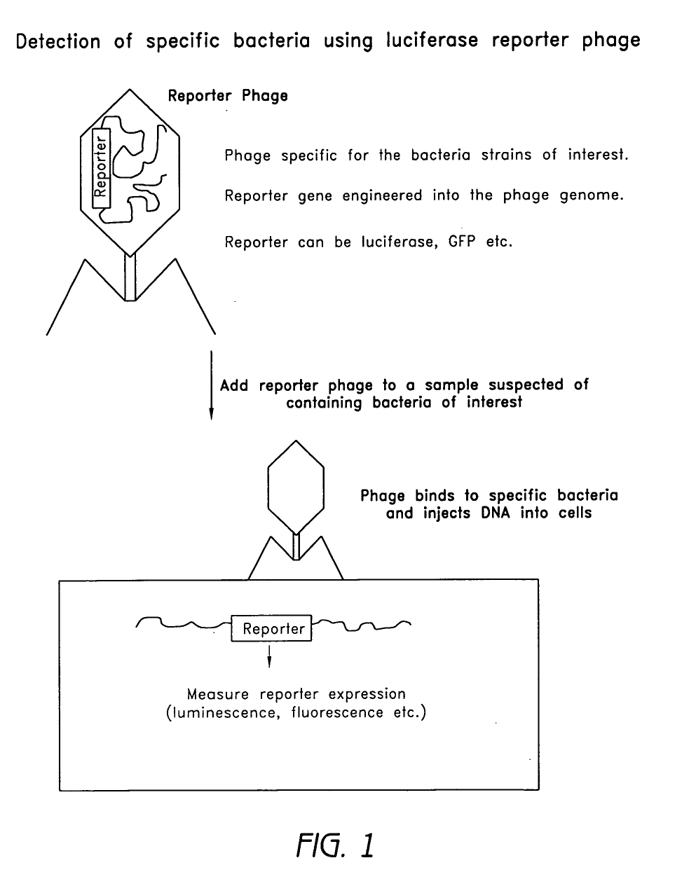

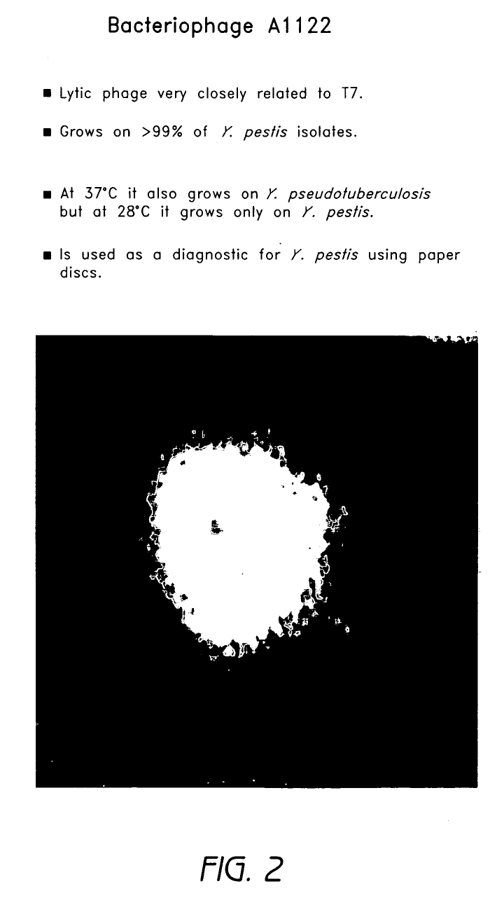

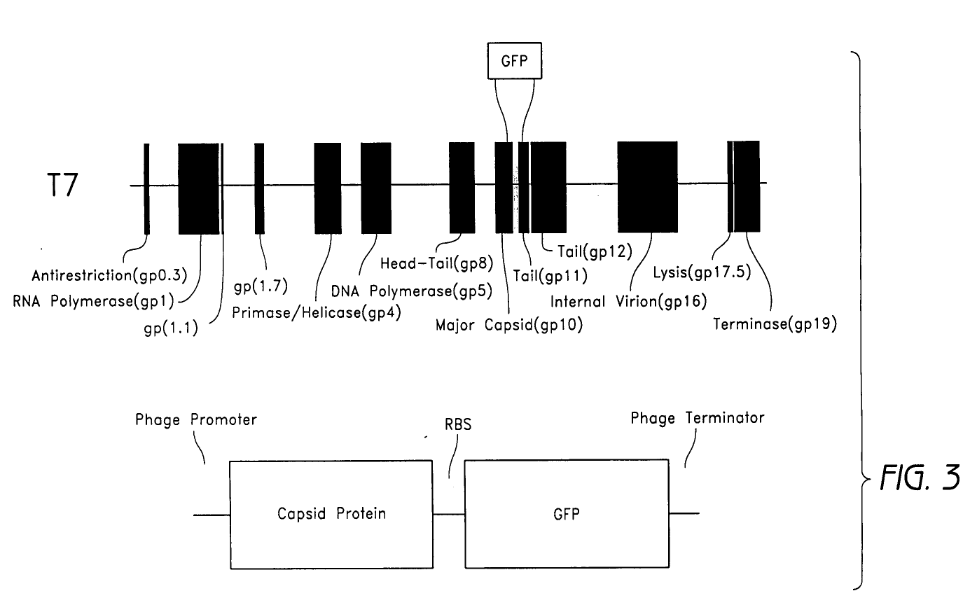

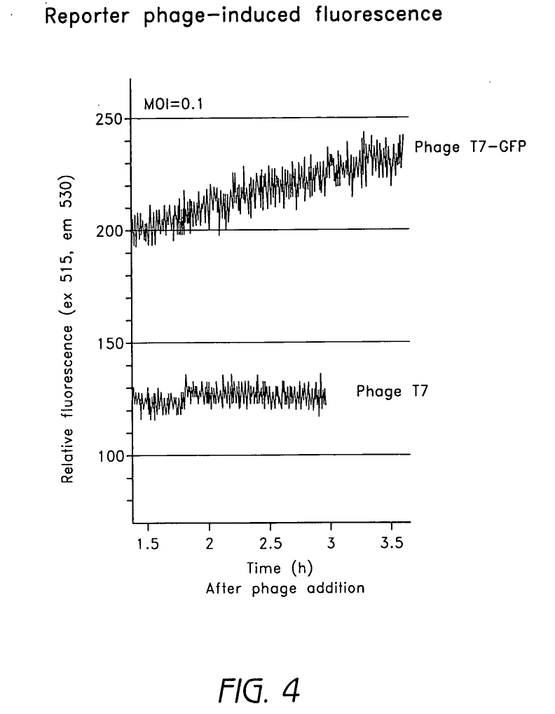

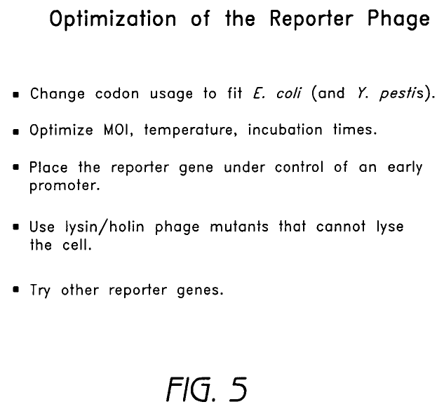

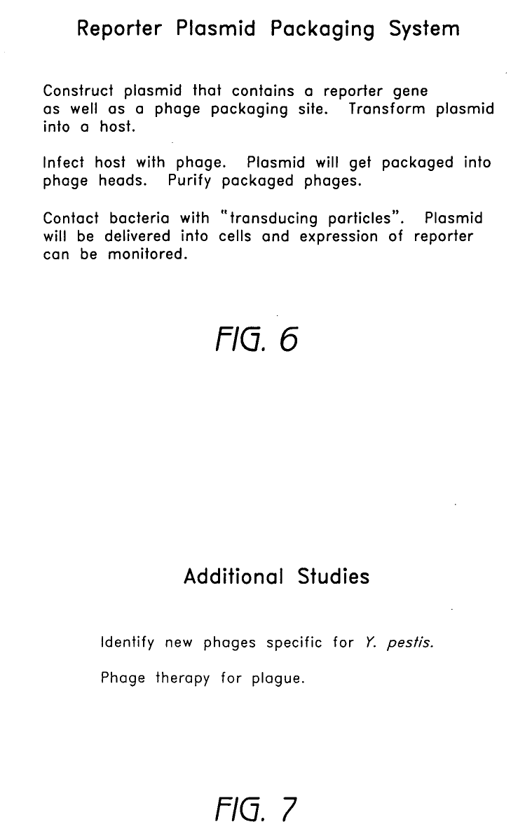

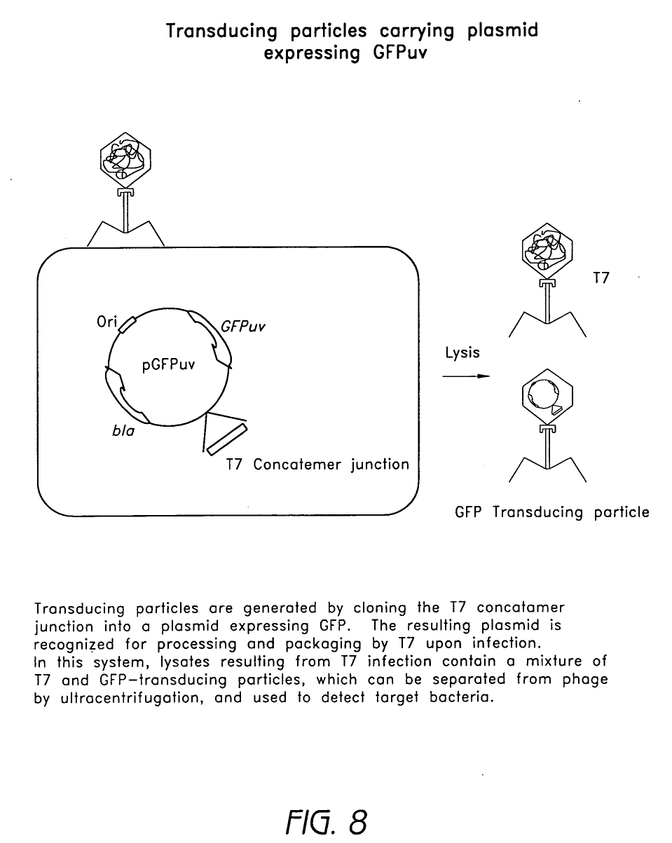

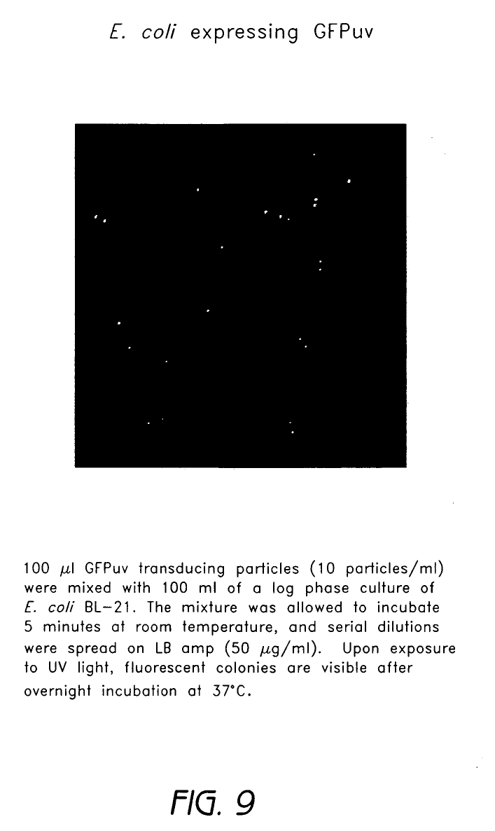

### DETAILED DESCRIPTION OF THE PREFERRED EMBODIMENT

The present invention utilizes modified bacteriophage, referred to hereinafter as “transducing particles”, in order to detect or identify bacterial cells in biological samples. As used herein, the term transducing particles shall also include component parts of a modified bacteriophage which component parts, when mixed together under proper conditions, will combine to form the modified bacteriophage. Transducing particles are comprised of a phage capsid and a plasmid DNA core. The plasmid DNA core comprises a reporter gene under the control of a bacteria-specific promoter, a bacteriophage packaging site wherein the packaging site is substantially in isolation from sequences naturally occurring adjacent thereto in the bacteriophage genome, a bacterial-specific origin of replication (ori), and optionally an antibiotic resistance marker gene that enables growth on selection media. The biological samples may be virtually any substance or medium capable of supporting bacterial growth or otherwise suspending bacterial cells in a viable state. Biological samples of particular interest to the present invention include water, soil, food samples, such as meat products and dairy products which are particularly susceptible to bacterial contamination, patient samples, such as blood, plasma, serum, sputum, semen, saliva, lavage, feces, cell culture, cerebrospinal fluid and the like.

Pathogenic Bacteria and Diseases they Cause

The range of bacterial cells to be detected is limited only by host ranges of available bacteriophages. Of particular interest are pathogenic bacteria which are capable of contaminating food and water supplies and are responsible for causing diseases in animals and man. Such pathogenic bacteria will usually be gram-negative, although the detection and identification of gram-positive bacteria is also a part of the present invention. A representative list of bacterial hosts of particular interest (with the diseases caused by such bacterial hosts) includes Actinomyces israelii (infection), Aeromonas hydrophila (gastroenteritis, septicemia), Bacillus anthracis (Anthrax: cutaneous, pulmonary), Bacillus subtilis (not considered pathogenic or toxigenic to humans, animals, or plants), Bacteriodes caccae (anaerobic infection), Bacteriodes distasonis (anaerobic infection), Bacteriodes merdae (anaerobic infection), Bacteriodes ovatus (anaerobic infection), Bacteriodes vulgatus (anaerobic infection), Bacteroides fragilis (anaerobic infection), Bacteroides thetaiotaomicron (anaerobic infection), Bordetella pertussis (Whooping cough), Borrelia burgdorferi (Lyme Disease), Brucella abortus (Brucellosis-cattle), Brucella canis (Brucellosis-dogs), Brucella melitensis (Brucellosis-sheep and goats), Brucella suis (Brucellosis-hogs), Burkholderia pseudomallei (infection: acute pulmonary, disseminated septicemic, nondisseminated septicemic, localized chronic suppurative), Campylobacter coli (diarrhea), Campylobacter fetus (bacteremia), Campylobacter jejuni (fever, abdominal cramps, and diarrhea, Guillain-Barre syndrome), Chlamydia trachomatis (Chlamydia), Clostridium botulinum (botulism), Clostridium butyricum (neonatal necrotizing enterocolitis, NEC), Clostridium difficile (NEC), Clostridium perfringes (myonecrosis-gas gangrene), clostridial cellulites, clostridial myositis, food disease, NEC), Clostridium tetani (tetanus), Corynebacterium diphtheriae (diptheria), Enterococcus durans (infection), Enterococcus faecalis (nosocomial infection), Enterococcus faecium (nosocomial infection), Erysipelothrix rhusiopathiae (erysipelothricosis), Escherichia coli (inflammatory or bloody diarrhea, urinary infection, bacteremia, meningitis), Francisella tularenisis (tularemia), the genus Fusobacterium (anaerobic infection), Haemophilus aegyptius (mucopurulent conjunctivitis, bacteremic Brazilian purpuric fever), Haemophilus aphrophilus (bacteremia, endocarditis and brain abscess), Haemophilus ducreyi (chancroid venereal disease), Haemophilus influenzae (bacterial meningitis, bacteremia, septic arthritis, pneumonia, tracheobronchitis, otitis media, conjunctivitis, sinusitis, acute epiglottitis, endocarditis), Heaemophilus parainfluenzae (bacteremia, endocarditis and brain abscess), Helicobacter pylori (gastric and duodenal ulcers, gastric cancers), Klebsiella pneumoniae (respiratory, urinary infection), Legionella pneumonphila (Legionaire's disease), the genus Leptospira (leptospirosis, or infectious spirochetal jaundice), Listeria ivanovii (listeriosis), Listeria monocylogenes (listeriosis), Listeria seeligeri (listeriosis), Morganella morganii (infection), Mycobacterium africanum (tuberculosis), Mycobacterium avium-intracellulare (Lady Windermere syndrome, mycobacterium avium complex, MAC), Mycobacterium bovis (tuberculosis), Mycobacterium chelonei (infection), Mycobacterium fortuitum (infection), Mycobacterium kansasii (infection), Mycobacterium leprae (leprosy), Mycobacterium marinum (infection), Mycobacterium tuberculosis (tuberculosis), Mycobacterium ulcerans (infection), Mycobacterium xenopi (infection), Neisseria gonorrhoeae (gonorrhea), Neisseria meningitidis (meningitis), Nocardia asteroids (nocardiosis), Prevotella melaninogenica (anaerobic infection), Proteus mirabilis (infection), Proteus mysofaciens (infection), Proteus vulgaris (infection), Providencia alcalifaciens (infection), Providencia rettgeri (infection), Providencia stuartii (infection), Pseudomonas acidovorans (nosocomial infection), Pseudomonas aeruginosa (nosocomial infection, i.e. in cystic fibrosis patients, burn victims, patients with permanent catheters), Pseudomonas fluorescens (nosocomial infection), Pseudomonas paucimobilis (nosocomial infection), Psuedomonas putida (nosocomial infection), Rickettsia rickettsti (Rocky Mountain spotted fever), Salmonella anatum (gastroenteritis, septicemia), Salmonella bovismorbificans (gastroenteritis, septicemia), Salmonella choleraesuis (gastroenteritis, septicemia), Salmonella Dublin (gastroenteritis, septicemia), Salmonella enteritidis (gastroenteritis, septicemia, enteric fever, bacteremia), Salmonella hirschifeldii (enteric fever), Salmonella Newington (gastroenteritis, septicemia), Salmonella paratyphi (paratyphoid), Salmonella schottmulleri (gastroenteritis, septicemia), Salmonella shottmuelleri (enteric fever), Salmonella typhi (typhoid fever), Serratia marcescens (wound infections), Shigella boydii (shigellosis), Shigella dysenteriae (shigellosis), Shigella flexneri (shigellosis), Shigella sonnei (shigellosis), Spirillum minus (rat-bite fever), Staphylococcus aureus (infections, food poisoning, toxic shock syndrome, pneumonia, bacteremia, endocarditis osteomyelitis enterocolitis, subcutaneous abscesses, exfoliation, meningitis), Streptobacillus moniliformis (rat-bite fever), Streptococcus agalactiae (neonatal sepsis, postpartum sepsis, endocarditis, and septic arthritis), Streptococcus antinosis (invasive infections), Streptococcus bovis (bacterial endocarditis), Streptococcus constellatus (invasive infections), Streptococcus iniae (cellulitis and invasive infections), Streptococcus intermedius (invasive infections), Streptococcus mitior (bacterial endocarditis), Streptococcus mutans (endocarditis), Streptococcus pneumoniae (pneumonia, acute otitis media, infection of the paranasal sinuses, acute purulent meningitis, bacteremia, pneumococcal endocarditis, pneumococcal arthritis, pneumococcal peritonitis), Streptococcus pyogenes (pharyngitis, tonsillitis, wound and skin infections, septicemia, scarlet fever, pneumonia, rheumatic fever and glomerulonephritis), Streptococcus salivarius (bacterial endocarditis), Streptococcus sanguis (bacterial endocarditis), Treponema palladum (syphilis), Vibrio alginolyticus (diarrhea, infection), Vibrio cholerae (cholera), Vibrio hollisae (diarrhea, infection), Vibrio mimicus (diarrhea, infection), Vibrio parahaemolyticus (diarrhea, infection), Vibrio vulnificus (diarrhea, infection), and Yersinia pestis (plague). The invention may also be used to detect subspecies of bacteria, for example E. coli 0157:H7.

### Bacteria and Corresponding, Host-Specific Bacteriophage

The range of bacterial cells to be detected is limited only by host ranges of available bacteriophages. A list of bacteria and corresponding, host-specific bacteriophages can be found on the internet at mansfield.ohio-state.edu/˜sabedon/names.htm. A listing of pathogenic bacterial genera and their known host-specific bacteriophages is presented in the following paragraphs. Synonyms and spelling variants are indicated in parentheses. Homonyms are repeated as often as they occur (e.g., D, D, d). Unnamed phages are indicated by “NN” beside their genus and their numbers are given in parentheses.

Bacteria of the genus Actinomyces are infected by the following phage: Av-1, Av-2, Av-3, BF307, CT1, CT2, CT3, CT4, CT6, CT7, CT8 and 1281.

Bacteria of the genus Aeromonas are infected by the following phage: AA-1, Aeh2, N, PM1, TP446, 3, 4, 11, 13, 29, 31, 32, 37, 43, 43-10T, 51, 54, 55R 1, 56, 56RR2, 57, 58, 59.1, 60, 63, Aeh1, F, PM2, 1, 25, 31, 40RR2.8t, (syn=44R), (syn=44RR_{2.8}t), 65, PM3, PM4, PM5 and PM6.

Bacteria of the genus Bacillus are infected by the following phage: A, aiz1, Al-K-I, B, BCJA1, BC1, BC2, BLL1, BL1, BP142, BSL1, BSL2, BS1, BS3, BS8, BS15, BS18, BS22, BS26, BS28, BS31, BS104, BS105, BS106, BTB, B1715V1, C, CK-1, Col1, Cor1, CP-53, CS-1, CS₁, D, D, D, D5, ent1, FP8, FP9, PS₁, FS₂, FS₃, FS₅, FS₈, FS₉, G, GH8, GT8, GV-1, GV-2, GT-4, g3, g12, g13, g14, g16, g17, g21, g23, g24, g29, H2, ken1, KK-88, Kum1, Kyu1, J7W-1, LP52, (syn=LP-52), L₇, Mex1, MJ-1, mor2, MP-7, MP10, MP12, MP14, MP15, Neo1, No 2, N5, N6P, PBC1, PBLA, PBP1, P2, S-a, SF2, SF6, Sha1, Sil1, SPO2, (syn=ΦSPP1), SPβ, STI, ST₁, SU-11, t, Tb1, Tb2, Tb5, Tb10, Tb26, Tb51, Tb53, Tb55, Tb77, Tb97, Tb99, Tb560, Tb595, Td8, Td6, Td15, Tg1, Tg4, Tg6, Tg7, Tg9, Tg10, Tg11, Tg13, Tg15, Tg21, Tin1, Tin7, Tin8, Tin13, Tm3, Toc1, Tog1, tol1, TP-1, TP-10ᵥᵢᵣ, TP-15c, TP-16c, TP-17c, TP-19, TP35, TP51, TP-84, Tt4, Tt6, type A, type B, type C, type D, type E, Tφ3, VA-9, W, wx23, wx26, Yun1, α, γ, ρ11, φmed-2, φT, φμ-4, φ3T, φ75, φ105, (syn=φ105), 1A, 1B, 1-97A, 1-97B, 2, 2, 3, 3, 3, 5, 12, 14, 20, 30, 35, 36, 37, 38, 41C, 51, 63, 64, 138D, I, II, IV, NN-Bacillus (13), ale1, AR1, AR2, AR3, AR7, AR9, Bace-11, (syn=11), Bastille, BL1, BL2, BL3, BL4, BL5, BL6, BL8, BL9, BP124, BS28, BS80, Ch, CP-51, CP-54, D-5, dar1, den1, DP-7, ent2, FoS₁, FoS₂, FS₄, FS₆, FS₇, G, gal1, gamma, GE1, GF-2, GS₁, GT-1, GT-2, GT-3, GT-4, GT-5, GT-6, GT-7, GV-6, g15, I9, I10, IS₁, K, MP9, MP13, MP21, MP23, MP24, MP28, MP29, MP30, MP32, MP34, MP36, MP37, MP39, MP40, MP41, MP43, MP44, MP45, MP47, MP50, NLP-1, No. 1, N17, N19, PBS1, PK1, PMB1, PMB12, PMJ1, S, SPO1, SP3, SP5, SP6, SP7, SP8, SP9, SP10, SP-15, SP50, (syn=SP-50), SP82, SST, sub1, SW, Tg8, Tg12, Tg13, Tg14, thu1, thu4, thu5, Tin4, Tin23, TP-13, TP33, TP50, TSP-1, type V, type VI, V, Vx, β22, φe, φNR2, φ25, φ63, 1, 1, 2, 2C, 3NT, 4, 5, 6, 7, 8, 9, 10, 12, 12, 17, 18, 19, 21, 138, III, 4 (B. megaterium), 4 (B. sphaericus), AR13, BPP-10, BS32, BS107, B1, B2, GA-I, GP-10, GV-3, GV-5, g8, MP20, MP27, MP49, Nf, PP5, PP6, SF5, Tg18, TP-1, Versailles, φ15, φ29, 1-97, 837/IV, NN-Bacillus (1), Bat10, BSL10, BSL11, BS6, BS11, BS16, BS23, BS101, BS102, g18, mor1, PBL1, SN45, thu2, thu3, Tm1, Tm2, TP-20, TP21, TP52, type F, type G, type IV, NN-Bacillus (3), BLE, (syn=θc), BS2, BS4, BS5, BS7, B10, B12, BS20, BS21, F, MJ-4, PBA12, AP50, AP50-04, AP50-11, AP50-23, AP50-26, AP50-27 and Bam35. The following Bacillus-specific phage are defective: DLP10716, DLP-11946, DPB5, DPB12, DPB21, DPB22, DPB23, GA-2, M, No. 1M, PBLB, PBSH, PBSV, PBSW, PBSX, PBSY, PBSZ, phi, SPα, type 1 and μ.

Bacteria of the genus Bacteriodes are infected by the following phage: ad1₂, Baf-44, Baf-48B, Baf-64, Bf-1, Bf-52, B40-8, F1, β1, φA1, φBr01, φBr02, 11, 67.1, 67.3, 68.1, NN-Bacteroides (3), Bf42, Bf71, NN-Bdellovibrio (1) and BF-41.

Bacteria of the genus Bordetella are infected by the following phage: 134 and NN-Bordetella (3).

Bacteria of the genus Borrellia are infected by the following phage: NN-Borrelia (1) and NN-Borrelia (2).

Bacteria of the genus Brucella are infected by the following phage: A422, Bk, (syn=Berkeley), BM₂₉, FO₁, (syn=FO1), (syn=FQ1), D, FP₂, (syn=FP2), (syn=FD2), Fz, (syn=Fz75/13), (syn=Firenze 75/13), (syn=Fi), F₁, (syn=F1), F₁m, (syn=F1m), (syn=Fim), F₁U, (syn=F1U), (syn=FiU), F₂, (syn=F2), F₃, (syn=F3), F4, (syn=F4), F₅, (syn=F5), F₆, F₇, (syn=F7), F₂₅, (syn=F25), (syn=f25), F₂₅U, (syn=F₂₅u), (syn=F25U), (syn=F25V), F₄₄, (syn=F44), F₄₅, (syn=F45), F₄₈, (syn=F48), I, Im, M, MC/75, M51, (syn=M85), P, (syn=D), S708, R, Tb, (syn=TB), (syn=Tbilisi), W, (syn=Wb), (syn=Weybridge), X, 3, 6, 7, 10/1, (syn=10), (syn=F₈), (syn=F8), 12m, 24/II, (syn=24), (syn=F₉), (syn=F9), 45/III, (syn=45), 75, 84, 212/XV, (syn=212), (syn=F₁₀), (syn=F10), 371/XXIX, (syn=371), (syn=F₁₁), (syn=P11) and 513.

Bacteria of the genus Burkholderia are infected by the following phage: CP75, NN-Burkholderia (1) and 42.

Bacteria of the genus Campylobacter are infected by the following phage: C type, NTCC12669, NTCC12670, NTCC12671, NTCC12672, NTCC12673, NTCC12674, NTCC12675, NTCC12676, NTCC12677, NTCC12678, NTCC12679, NTCC12680, NTCC12681, NTCC12682, NTCC12683, NTCC12684, 32f, 111c, 191, NN-Campylobacter (2), Vfi-6, (syn=V19), Vfv-3, V2, V3, V8, V16, (syn=Vfi-1), V19, V20(V45), V45, (syn=V-45) and NN-Campylobacter (1).

Bacteria of the genus Chlamydia are infected by the following phage: Chp1.

Bacteria of the genus Clostridium are infected by the following phage: CAK1, CA5, Ca7, CEβ, (syn=1C), CEγ, Cld1, c-n71, c-203 Tox-, DEβ, (syn=1D), (syn=1D^{tox+}), HM3, KM1, KT, Ms, NA1, (syn=Na1^{tox+}), PA1350e, Pfô, PL73, PL78, PL81, P1, P50, P5771, P19402, 1C^{tox+}, 2C^{tox-}, 2D, (syn=2D^{tox+}), 3C, (syn=3C^{tox+}), 4C, (syn=4^{tox+}), 56, III-1, NN-Clostridium (61), NB1^{tox-}α1, CA1, HMT, HM2, PF1, P-₂₃, P-₄₆, Q-₀₅, Q-₀₆, Q-₁₆, Q-₂₁, Q-₂₆, Q-₄₀, Q-₄₆, S₁₁₁, SA₀₂, WA₀₁, WA₀₃, W₁₁₁, W₅₂₃, 80, C, CA2, CA3, CPT1, CPT4, c1, c4, c5, HM7, H₁₁/A₁, H₁₈/A₁, H₂₂/S₂₃, H₁₅₈/A₁, K₂/A₁, K₂₁/S₂₃, M_{L}, NA2^{tox-}, Pf2, Pf3, Pf4, S₉/S₃, S₄₁/A₁, S₄₄/S₂₃, α2, 41, 112/S₂₃, 214/S₂₃, 233/A₁, 234/S₂₃, 235/S₂₃, II-1, II-2, II-3, NN-Clostridium (12), CA1, F1, K, S2, 1, 5 and NN-Clostridium (8).

Bacteria of the genus Corynebacterium are infected by the following phage: CGI1 (defective), A, A2, A3, A110, A128, A133, A137, A139, A155, A182, B, BF, B17, B18, B51, B271, B275, B276, B277, B279, B282, C, cap₁, CC1, CG1, CG2, CG33, CL31, Cog, (syn=CG5), D, E, F, H, H-1, hq₁, hq₂, I₁/H₃₃, I₁/H₃₃, J, K, K, (syn=K^{tox-}), L, L, (syn=L^{tox+}), M, MC-1, MC-2, MC-3, MC-4, MLMa, N, O, ov₁, ov₂, ov₃, P, P, P, RP6, R_{S}29, S, T, U, UB₁, ub₂, UH₁, UH₃, uh₃, uh₅, uh₆, β, (syn=β^{tox+}), β^{lav64}, βvir, γ, (syn=γ^{tox-}), γ19, δ, (syn=δ^{tox+}), ρ, (syn=ρ^{tox-}), φ9, φ984, ω, 1A, 1/1180, 2, 2/1180, 5/1180, 5ad/9717, 7/4465, 8/4465, 8ad/10269, 10/9253, 13/9253, 15/3148, 21/9253, 28, 29, 55, 2747, 2893, 4498 and 5848.

Bacteria of the genus Enterococcus are infected by the following phage: DF₇₈, F1, F2, 1, 2, 4, 14, 41, 867, D1, SB24, 2BV, 182, 225, C2, C2F, E3, E62, DS96, H24, M35, P3, P9, SB101, S2, 2BII, 5, 182a, 705, 873, 881, 940, 1051, 1057, 21096C, NN-Enterococcus (1), PE1, P1, F3, F4, VD13, 1, 200, 235 and 341.

Bacteria of the genus Erysipelothrix are infected by the following phage: NN-Erysipelothrix (1).

Bacteria of the genus Escherichia are infected by the following phage: BW73, B278, D6, D108, E, E1, E24, E41, FI-2, FI-4, FI-5, HI8A, HI8B, i, MM, Mu, (syn=mu), (syn=Mu1), (syn=Mu-1), (syn=MU-1), (syn=MuI), (syn=mu), O25, PhI-5, Pk, PSP3, P1, P1D, P2, P4 (defective), S1, Wφ, φK13, φR73 (defective), φ1, φ2, φ7, φ92, ψ (defective), 7A, 8φ, 9φ, 15 (defective), 18, 28-1, 186, 299, NN-Escherichia (2), AB48, CM, C4, C16, DD-VI, (syn=D_{d}−Vi), (syn=DDVI), (syn=DDVi), E4, E7, E28, F11, F13, H, H1, H3, H8, K3, M, N, ND-2, ND-3, ND4, ND-5, ND6, ND-7, Ox-1, (syn=OX1), (syn=11F), Ox-2, (syn=Ox2), (syn=OX2), Ox-3, Ox-4, Ox-S, (syn=OX5), Ox-6, (syn=66F), (syn=φ66t), (syn=φ66t-), O111, PhI-1, RB42, RB43, RB49, RB69, S, Sal-1, Sal-2, Sal-3, Sal-4, Sal-5, Sal-6, TC23, TC45, TuII*-6, (syn=TuII*), TuII*-24, TuII*46, TuII*-60, T2, (syn=gamma), (syn=γ), (syn=PC), (syn=P.C.), (syn=T-2), (syn=T₂), (syn=P₄), T4, (syn=T-4), (syn=T₄), T6, T35, α1, 1, 1A, 3, (syn=Ac3), 3A, 3T⁺, (syn=3), (syn=M1), 5φ, (syn=φ5), 9266Q, CFO103, HK620, J, K, K1F, m59, no. A, no. E, no. 3, no. 9, N4, sd, (syn=Sd), (syn=S_{D}), (syn=S_{d}), (syn=s_{d}), (syn=SD), (syn=CD), T3, (syn=T-3), (syn=T₃), T7, (syn=T-7), (syn=T₇), WPK, W31, Δ^{H}, φC3888, φK3, φK7, φK12, φV-1, Φ04-CF, Φ05, Φ06, Φ07, φ1, φ1.2, φ20, φ95, φ263, φ1092, φI, φII, (syn=φW), Ω8, 1, 3, 7, 8, 26, 27, 28-2, 29, 30, 31, 32, 38, 39, 42, 933W, NN-Escherichia (1), Esc-7-11, AC30, CVX-5, C1, DDUP, EC1, EC2, E21, E29, F1, F26S, F27S, Hi, HK022, HK97, (syn=ΦHK97), HK139, HK253, HK256, K7, ND-1, no. D, PA-2, q, S2, T1, (syn=α), (syn=P28), (syn=T-1), (syn=T₁), T3C, T5, (syn=T-5), (syn=T₅), UC-1, w, β4, γ2, λ, (syn=lambda), (syn=Φλ), ΦD326, φγ, Φ06, Φ7, Φ10, φ80, χ, (syn=χ₁), (syn=φχ), (syn=φχ₁), 2, 4, 4A, 6, 8A, 102, 150, 168, 174, 3000, AC6, AC7, AC28, AC43, AC50, AC57, AC81, AC95, HK243, K10, ZG/3A, 5, 5A, 21EL, H19-f and 933H.

Bacteria of the genus Fusobacterium are infected by the following phage: NN-Fusobacterium (2), fv83-554/3, fv88-531/2, 227, fv2377, fv2527 and fv8501.

Bacteria of the genus Haemophilus are infected by the following phage: HP1, S2 and N3.

Bacteria of the genus Helicobacter are infected by the following phage: HP1 and NN-Helicobacter (1).

Bacteria of the genus Klebsiella are infected by the following phage: AIO-2, Kl₄B, Kl₆B, Kl₉, (syn=Kl9), Kl14, Kl₁₅, Kl21, Kl28, Kl₂₉, Kl₃₂, Kl₃₃, Kl₃₅, Kl₁₀₆B, Kl₁₇₁B, Kl₁₈₁B, Kl₈₃₂B, AIO-1, AO-1, AO-2, AO-3, FC3-10, K, Kl₁, (syn=Kl1), Kl₂, (syn=K12), Kl₃, (syn=Kl3), (syn=K170/11), Kl₄, (syn=Kl4), Kl₅, (syn=Kl5), Kl₆, (syn=Kl6), Kl₇, (syn=Kl7), Kl₈, (syn=K₁₈), Kl₁₉, (syn=Kl9), Kl₂₇, (syn=K127), Kl₃₁, (syn=Kl31), Kl₃₅, Kl₁₇₁B, II, VI, IX, CI-1, Kl₄B, Kl₈, Kl₁₁, Kl₁₂, Kl₁₃, Kl₁₆, Kl₁₇, Kl₁₈, Kl₂₀, Kl₂₂, Kl₂₃, Kl₂₄, Kl₂₆, Kl₃₀, Kl₃₄, Kl₁₀₆B, Kl₁₆₅B, Kl₃₂₈B, KLXI, K328, P5046, 11, 380, III, IV, VII, VIII, FC3-11, Kl₂B, (syn=Kl2B), Kl₂₅, (syn=Kl25), Kl₄₂B, (syn=Kl42), (syn=Kl42B), Kl₁₈₁B, (syn=Kl181), (syn=Kl181B), Kl_{765/1}, (syn=Kl765/1), Kl₈₄₂B, (syn=Kl832B), Kl₉₃₇B, (syn=Kl937B), L1, φ28, 7, 231, 483, 490, 632 and 864/100.

Bacteria of the genus Lepitospira are infected by the following phage: LE1, LE3, LE4 and NN-Leptospira (1).

Bacteria of the genus Listeria are infected by the following phage: A511, O1761, 4211, 4286, (syn=BO54), A005, A006, A020, A500, A502, A511, A118, A620, A640, B012, B021, B024, B025, B035, B051, B053, B054, B055, B056, B101, B110, B545, B604, B653, C707, D441, HSO47, H1OG, H8/73, H19, H21, H43, H46, H107, H108, H10, H163/84, H1312, H340, H387, H391/73, H684/74, H924A, PSA, U153, φMLUP5, (syn=P35), 00241, 00611, 02971A, 02971C, 5/476, 5/911, 5/939, 5/11302, 5/11605, 5/11704, 184, 575, 633, 699/694, 744, 900, 1090, 1317, 1444, 1652, 1806, 1807, 1921/959, 1921/11367, 1921/11500, 1921/11566, 1921/12460, 1921/12582, 1967, 2389, 2425, 2671, 2685, 3274, 3550, 3551, 3552, 4276, 4277, 4292, 4477, 5337, 5348/11363, 5348/11646, 5348/12430, 5348/12434, 10072, 11355C, 11711A, 12029, 12981, 13441, 90666, 90816, 93253, 907515, 910716 and NN-Listeria (15).

Bacteria of the genus Morganella are infected by the following phage: 47.

Bacteria of the genus Mycobacterium are infected by the following phage: I3, AG1, AL₁, ATCC 11759, A2, B.C₃, BG2, BK1, BK₅, butyricum, B-1, B5, B7, B30, B35, Clark, C1, C2, DNAIII, DSP₁, D4, D29, GS4E, (syn=GS₄E), GS7, (syn=GS-7), (syn=GS₇), IPα, lacticola, Legendre, Leo, L5, (syn=ΦL-5), MC-1, MC-3, MC-4, minetti, MTPH11, Mx4, MyF₃P/59a, phlei, (syn=phlei 1), phlei 4, Polonus II, rabinovitschi, smegmatis, TM4, TM9, TM10, TM120, Y7, Y10, φ630, 1B, 1F, 1H, 1/1, 67, 106, 1430, B1, (syn=Bol), B₂₄, D, D29, F-K, F-S, HP, Polonus I, Roy, R1, (syn=R1-Myb), (syn=R₁), 11, 31, 40, 50, 103a, 103b, 128, 3111-D, 3215-D and NN-Mycobacterium (1).

Bacteria of the genus Neisseria are infected by the following phage: Group I, group II and NP1.

Bacteria of the genus Nocardia are infected by the following phage: P8, NJ-L, NS-8, N5 and NN-Nocardia (1).

Bacteria of the genus Proteus are infected by the following phage: Pm5, 13vir, 2/44, 4/545, 6/1004, 13/807, 20/826, 57, 67b, 78, 107/69, 121, 9/0, 22/608, 30/680, Pm1, Pm3, Pm4, Pm6, Pm7, Pm9, Pm10, Pm11, Pv2, π1, φm, 7/549, 9B/2, 10A/31, 12/55, 14, 15, 16/789, 17/971, 19A/653, 23/532, 25/909, 26/219, 27/953, 32A/909, 33/971, 34/13, 65, 5006M, 7480b, VI, 13/3a, Clichy 12, π2600, φχ7, 1/1004, 5/742, 9, 12, 14, 22, 24/860, 2600/D52, Pm8 and 24/2514.

Bacteria of the genus Providencia are infected by the following phage: PL25, PL26, PL37, 9211/9295, 9213/9211b, 9248, 7/R49, 74761322, 7478/325, 7479, 7480, 9000/9402 and 9213/9211a.

Bacteria of the genus Pseudomonas are infected by the following phage: Pf1, (syn=Pf-1), Pf2, Pf3, PP7, PRR1, 7s, NN-Pseudomonas (1), AI-1, M-2, B17, B89, CB3, Col 2, Col 11, Col 18, Col 21, C154, C163, C167, C2121, E79, F8, ga, gb, H22, K₁, M4, N₂, Nu, PB-1, (syn=PB1), pf16, PMN17, PP1, PP8, Psa1, PsP1, PsP2, PsP3, PsP4, PsP5, PS3, PS17, PTB80, PX4, PX7, PYO1, PYO2, PYO5, PYO6, PYO9, PYO10, PYO13, PYO14, PYO16, PYO18, PYO19, PYO20, PYO29, PYO32, PYO33, PYO35, PYO36, PYO37, PYO38, PYO39, PYO41, PYO42, PYO45, PYO47, PYO48, PYO64, PYO69, PYO103, P1K, SLP1, SL2, S₂, UNL-1, wy, Ya₁, Ya₄, Ya₁₁, φBE, φCTX, φC17, φKZ, (syn=ΦKZ), φ-LT, Φmu78, φNZ, φPLS-1, φST-1, φW-14, φ-2, 1/72, 2/79, 3, 3/DO, 4/237, 5/406, 6C, 6/6660, 7, 7v, 7/184, 8/280, 9/95, 10/502, 11/DE, 12/100, 12S, 16, 21, 24, 25F, 27, 31, 44, 68, 71, 95, 109, 188, 337, 352, 1214, NN-Pseudomonas (23), A856, B26, CI-1, CI-2, C5, D, gh-1, F116, HF, H90, K₅, K₆, K104, K109, K166, K267, N₄, N₅, O6N-25P, PE69, Pf, PPN25, PPN35, PPN89, PPN91, PP2, PP3, PP4, PP6, PP7, PP8, PP56, PP87, PP114, PP206, PP207, PP306, PP651, Psp231a, Pssy401, Pssy9220, ps₁, PTB2, PTB20, PTB42, PX1, PX3, PX10, PX12, PX14, PYO70, PYO71, R, SH6, SH133, tf, Ya₅, Ya₇, φBS, ΦKf77, φ-MC, ΦmnF82, φPLS27, φPLS743, φS-1, 1, 2, 2, 3, 4, 5, 6, 7, 7, 8, 9, 10, 11, 12, 12B, 13, 14, 15, 14, 15, 16, 17, 18, 19, 20, 20, 21, 21, 22, 23, 23, 24, 25, 31, 53, 73, 119x, 145, 147, 170, 267, 284, 308, 525, NN-Pseudomonas (5), af, A7, B3, B33, B39, BI-1, C22, D3, D37, D40, D62, D3112, F7, F10, g, gd, ge, gf, Hw12, Jb19, KF1, L°, OXN-32P, O6N-52P, PCH-1, PC13-1, PC35-1, PH2, PH51, PH93, PH132, PMW, PM13, PM57, PM61, PM62, PM63, PM69, PM105, PM113, PM681, PM682, PO4, PP1, PP4, PP5, PP64, PP65, PP66, PP71, PP86, PP88, PP92, PP401, PP711, PP891, Pssy41, Pssy42, Pssy403, Pssy404, Pssy420, Pssy923, PS4, PS-10, Pz, SD1, SL1, SL3, SL5, SM, φC5, φC11, φC11-1, φC13, φC15, φMO, φX, φ04, φ11, φ240, 2, 2F, 5, 7m, 11, 13, 13/441, 14, 20, 24, 40, 45, 49, 61, 73, 148, 160, 198, 218, 222, 236, 242, 246, 249, 258, 269, 295, 297, 309, 318, 342, 350, 351, 357-1, 400-1, NN-Pseudomonas (6), G10, M6, M6a, L1, PB2, Pssy15, Pssy4210, Pssy4220, PYO12, PYO34, PYO49, PYO50, PYO51, PYO52, PYO53, PYO57, PYO59, PYO200, PX2, PX5, SL4, φ03, φ06 and 1214.

Bacteria of the genus Rickettsia are infected by the following phage: NN-Rickettsia (1).

Bacteria of the genus Salmonella are infected by the following phage: b, Beccles, CT, d, Dundee, f, Fels 2, GI, GIII, GVI, GVIII, k, K, i, j, L, O1, (syn=O-1), (syn=O₁), (syn=O-I), (syn=7), O2, O3, P3, P9a, P10, Sab3, Sab5, San15, San17, SI, Taunton, ViI, (syn=Vil), 9, NN-Salmonella (1), N-1, N-5, N-10, N-17, N-22, 11, 12, 16-19, 20.2, 36, 449C/C178, 966A/C259, a, B.A.O.R., e, G4, GIII, L, LP7, M, MG40, N-18, PSA68, P4, P9c, P22, (syn=P₂₂), (syn=PLT22), (syn=PLT₂₂), P22a1, P22-4, P22-7, P22-11, SNT-1, SNT-2, SP6, ViIII, ViIV, ViV, ViVI, ViVII, Worksop, ε₁₅, ε₃₄, 1, 37, 1(40), (syn=φ1[40]), 1, 42₂, 2, 2.5, 3b, 4, 5, 6, 14(18), 8, 14(6,7), 10, 27, 28B, 30, 31, 32, 33, 34, 36, 37, 39, 1412, SNT-3,7-11, 40.3, c, C236, C557, C625, C966N, g, GV, G5, G173, h, IRA, Jersey, M78, P22-1, P22-3, P22-12, Sab1, Sab2, Sab2, Sab4, San1, San2, San3, San4, San6, San7, San8, San9, San13, San14, San16, San18, San19, San20, San21, San22, San23, San24, San25, San26, SasL₁, SasL2, SasL3, SasL4, SasL5, S1BL, SII, ViII, φ1, 1, 2, 3a, 3aI, 1010, NN-Salmonella (1), N-4, SasL6 and 27.

Bacteria of the genus Serratia are infected by the following phage: A2P, PS20, SMB3, SMP, SMP5, SM2, V40, V56, κ, DCP-3, (DCP-6, 3M, 10/1a, 20A, 34CC, 34H, 38T, 345G, 345P, 501B, SMB2, SMP2, BC, BT, CW2, CW3, CW4, CW5, L₁232, L₂232, L34, L.228, SLP, SMPA, V.43, σ, φCW1, ΦCP6-1, ΦCP6-2, ΦCP6-5, 3T, 5, 8, 9F, 10/1, 20E, 32/6, 34B, 34CT, 34P, 37, 41, 56, 56D, 56P, 60P, 61/6, 74/6, 76/4, 101/8900, 226, 227, 228, 229F, 286, 289, 290F, 512, 764a, 2847/10, 2847/10a, L.359 and SMB1,

Bacteria of the genus Shigella are infected by the following phage: Fsa, (syn=a), FS_{D2d}, (syn=D2d), (syn=W₂d), FS_{D2E}, (syn=W₂e), fv, F6, f7.8, H-Sh, PE5, P90, SfII, Sh, SH_{III}, SH_{IV}, (syn=HIV), SH_{VI}, (syn=HVI), SH_{VIII}, (syn=HVIII), SKγ66, (syn=gamma 66), (syn=γ66), (syn=γ66b), SK_{III}, (syn=SIIIb), (syn=III), SK_{IV}, (syn=S_{IVa}), (syn=IV), SK_{IVa}, (syn=S_{IVAn}), (syn=IVA), SK_{VI}, (syn=KVI), (syn=S_{VI}), (syn=VI), SK_{VIII}, (syn=S_{VIII}), (syn=VIII), SK_{VIIIA}, (syn=S_{VIII}A), (syn=VIIIA), ST_{VI}, ST_{IX}, ST_{XI}, ST_{XII}, S66, W₂, (syn=D2c), (syn=D20), φI, φIV₁, 3-SO-R, 8368-SO-R, F7, (syn=FS7), (syn=K₂₉), F10, (syn=FS10), (syn=K31), I₁, (syn=alfa), (syn=FSα), (syn=K18), (syn=α), I₂, (syn=a), (syn=K19), SG₃₅, (syn=G35), (syn=SO-35/G), SG₅₅, (syn=SO-55/G), SG₃₂₀₁, (syn=SO-3201/G), SH_{II}, (syn=HII), SH_{V}, (syn=SHV), SH_{X}, SHX, SK_{II}, (syn=K2), (syn=KII), (syn=S_{II}), (syn=SsII), (syn=II), SK_{IV}, (syn=S_{IVb}), (syn=SsIV), (syn=IV), SK_{IVa}, (syn=S_{IVab}), (syn=SsIVa), (syn=IVa), SK_{V}, (syn=K4), (syn=KV), (syn=SV), (syn=SsV), (syn=V), SK_{X}, (syn=K₉), (syn=KX), (syn=SX), (syn=SsX), (syn=X), ST_{V}, (syn=T35), (syn=35-50-R), ST_{VIII}, (syn=T8345), (syn=8345-SO-S-R), W₁, (syn=D8), (syn=FS_{D8}), W₂a, (syn=D2A), (syn=FS₂ₐ), DD-2, Sf6, FS₁, (syn=F1), SF₆, (syn=F6), SG₄₂, (syn=SO-42/G), SG₃₂₀₃, (syn=SO-3203/G), SK_{F12}, (syn=SsF₁₂), (syn=F₁₂), (syn=F12), ST_{II}, (syn=1881-SO-R), γ66, (syn=gamma 66a), (syn=Ssγ66), φ2, B11, DDVII, (syn=DD7), FS_{D2b}, (syn=W₂B), FS₂, (syn=F₂), (syn=F2), FS₄, (syn=F₄), (syn=F4), FS₅, (syn=F₅), (syn=F5), FS₉, (syn=F₉), (syn=F9), F11, P2-SO-S, SG₃₆, (syn=SO-36/G), (syn=G36), SG₃₂₀₄, (syn=SO-3204/G), SG₃₂₄₄, (syn=SO-3244/G), SH_{I}, (syn=HI), SH_{VII}, (syn=HVII), SH_{IX}, (syn=HIX), SH_{XI}, SH_{XII}, (syn=HXII), SKI, KI, (syn=S_{I}), (syn=SsI), SKVII, (syn=KVII), (syn=S_{VII}), (syn=SsVII), SKIX, (syn=KIX), (syn=S_{IX}), (syn=SsIX), SKXII, (syn=KXII), (syn=S_{VII}), (syn=SsXII), ST_{I}, S_{III}, ST_{III}, ST_{IV}, ST_{VII}, S70, S206, U2-SO—S, 3210-SO-S, 3859-SO-S, 4020-SO-S, φ3, φ5, φ7, φ8, φ9, φ10, φ11, φ13, φ14, φ18, SH_{III}, (syn=HIII), SH_{XI}, (syn=HXI) and S_{XI}, (syn=KXI), (syn=S_{XI}), (syn=SsXI), (syn=XI).

Bacteria of the genus Staphylococcus are infected by the following phage: A, EW, K, Ph5, Ph9, Ph10, Ph13, P1, P2, P3, P4, P8, P9, P10, RG, S_{B-1}, (syn=Sb-1), S3K, Twort, φSK311, φ812, 06, 40, 58, 119, 130, 131, 200, 1623, STC1, (syn=stc1), STC2, (syn=stc2), 44AHJD, 68, AC1, AC2, A6″C″, A9″C″, b⁵⁸¹, CA-1, CA-2, CA-3, CA-4, CA-5, D11, L39×35, L₅₄a, M42, N1, N2, N3, N4, N5, N7, N8, N10, N11, N12, N13, N14, N16, Ph6, Ph12, Ph14, UC-18, U4, U15, S1, S2, S3, S4, S5, X2, Z₁, φB5-2, φD, ω, 11, (syn=φ11), (syn=P11-M15), 15, 28, 28A, 29, 31, 31B, 37, 42D, (syn=P42D), 44A, 48, 51, 52, 52A, (syn=P52A), 52B, 53, 55, 69, 71, (syn=P71), 71A, 72, 75, 76, 77, 79, 80, 80a, 82, 82A, 83A, 84, 85, 86, 88, 88A, 89, 90, 92, 95, 96, 102, 107, 108, 111, 129-26, 130, 130A, 155, 157, 157A, 165, 187, 275, 275A, 275B, 356, 456, 459, 471, 471A, 489, 581, 676, 898, 1139, 1154A, 1259, 1314, 1380, 1405, 1563, 2148, 2638A, 2638B, 2638C, 2731, 2792A, 2792B, 2818, 2835, 2848A, 3619, 5841, 12100, AC3, A8, A10, A13, b594n, D, M12, N9, N15, P52, P87, S1, S6, Z₄, φRE, 3A, 3B, 3C, 6, 7, 16, 21, 42B, 42C, 42E, 44, 47, 47A, 47C, 51, 54, 54×1, 70, 73, 75, 78, 81, 82, 88, 93, 94, 101, 105, 110, 115, 129/16, 174, 594n, 1363/14, 2460 and NN-Staphylococcus (1).

Bacteria of the genus Streptococcus are infected by the following phage: EJ-1, NN-Streptococcus (1), a, Cl, F_{LO}Ths, H39, Cp-1, Cp-5, Cp-7, Cp-9, Cp-10, AT298, A5, a10/J1, a10/J2, a10/J5, a10/J9, A25, BT11, b6, CA1, c20-1, c20-2, DP-1, Dp-4, DT1, ET42, e10, F_{A}101, F_{E}Ths, F_{K}, F_{KK}101, F_{KL}10, F_{KP}74, F_{K}11, F_{LO}Ths, F_{Y}101, f1, F₁₀, F₂₀140/76, g, GT-234, HB3, (syn=HB-3), HB-623, HB-746, M102, O1205, φO1205, PST, P0, P1, P2, P3, P5, P6, P8, P9, P9, P12, P13, P14, P49, P50, P51, P52, P53, P54, P55, P56, P57, P58, P59, P64, P67, P69, P71, P73, P75, P76, P77, P82, P83, P88, sc, sch, sf, Sfi11, (syn=SFi11), (syn=φSFi11), (syn=ΦSfi11), (syn=φSfi11), sfi19, (syn=SFi19), (syn=φSFi19), (syn=φSfi19), Sfi21, (syn=SFi21), (syn=φSFi21), (syn=φSfi21), ST_{G}, STX, st2, ST₂, ST₄, S3, (syn=φS3), s265, Φ17, φ42, Φ57, φ80, φ81, φ82, φ83, φ84, φ85, φ86, φ87, φ88, φ89, φ90, φ91, φ92, φ93, φ94, φ95, φ96, φ97, φ98, φ99, φ100, φ101, φ102, φ227, Φ7201, ω1, ω2, ω3, ω4, ω5, ω6, ω8, ω10, 1, 6, 9, 10F, 12/12, 14, 17SR, 19S, 24, 50/33, 50/34, 55/14, 55/15, 70/35, 70/36, 71/ST15, 71/45, 71/46, 74F, 79137, 79/38, 80/J4, 80/J9, 80/ST16, 80/15, 80/47, 80/48, 101, 103/39, 103/40, 121/41, 121/42, 123/43, 123/44, 124/44, 337/ST17 and NN-Streptococcus (34).

Bacteria of the genus Treponema are infected by the following phage: NN-Treponema (1).

Bacteria of the genus Vibrio are infected by the following phage: CTXΦ, fs, (syn=s1), fs2, 1vpfs, Vf12, Vf33, VPIΦ, VSK, v6, 493, CP-T1, ET25, kappa, K139, LaboI,) XN-69P, OXN-86, O6N-21P, PB-1, P147, rp-1, SE3, VA-1, (syn=VcA-1), VcA-2, VcA-1, VP1, VP2, VP4, VP7, VP8, VP9, VP10, VP17, VP18, VP19, X29, (syn=29 d'Hérelle), 1, ΦHAWI-1, ΦHAWI-2, ΦHAWI-3, ΦHAWI-4, ΦHAWI-5, ΦHAWI-6, ΦHAWI-7, ΦHAWI-8, ΦHAWI-9, ΦHAWI-10, ΦHC1-1, ΦHCl-2, ΦHCl-3, ΦHC1-4, ΦHC2-1, ΦHC2-2, ΦHC2-3, ΦHC2-4, ΦHC3-1, ΦHC3-2, ΦHC3-3, ΦHD1S-1, ΦHD1S-2, ΦHD2S-1, ΦHD2S-2, ΦHD2S-3, ΦHD2S-4, ΦHD2S-5, ΦHDO-1, ΦHDO-2, ΦHDO-3, ΦHDO-4, ΦHDO-5, ΦHDO-6, ΦKL-33, ΦKL-34, ΦKL-35, ΦKL-36, ΦKW1H-2, ΦKWH-3, ΦKWH-4, ΦMARQ-1, ΦMARQ-2, ΦMARQ-3, ΦMOAT-1, ΦO139, ΦPEL1A-1, ΦPEL1A-2, ΦPEL8A-1, ΦPEL8A-2, ΦPEL8A-3, ΦPEL8C-1, ΦPEL8C-2, ΦPEL13A-1, ΦPEL-13B-1, ΦPEL13B3-2, ΦPEL13B-3, ΦPEL13B-4, ΦPEL13B-5, ΦPEL13B-6, ΦPEL13B-7, ΦPEL13B-8, ΦPEL13B-9, ΦPEL13B-10, φVP143, φVP253, Φ16, φ138, 1-11, 5, 13, 14, 16, 24, 32, 493, 6214, 7050, 7227, II, (syn=group 11), (syn=φ2), V, VIII, NN-Vibrio (13), KVP20, KVP40, nt-1, O6N-22P, P68, e1, e2, e3, e4, e5, FK, G, J, K, nt-6, N1, N2, N3, N4, N5, O6N-34P, OXN-72P, OXN-85P, OXN-100P, P, Ph-1, PL163/10, Q, S, T, φ92, 1-9, 37, 51, 57, 70A-8, 72A-4, 72A-10, 110A-4, 333, 4996, I, (syn=group I), III, (syn=group III), VI, (syn=A-Saratov), VII, IX, X, NN-Vibrio (6), pA1, 7, 7-8, 70A-2, 71A-6, 72A-5, 72A-8, 108A-10, 109A-6, 109A-8, 110A-1, 110A-5, 110A-7, hv-1, OXN-52P, P13, P38, P53, P65, P108, P111, TP1, VP3, VP6, VPI2, VP13, 70A-3, 70A-4, 70A-10, 72A-1, 108A-3, 109-B1, 110A-2, 149, (syn=φ149), IV, (syn=group IV), NN-Vibrio (22), VP5, VP11, VP15, VP16, α1, α2, α3a, 3b, 353B and NN-Vibrio (7).

Bacteria of the genus Yersinia are infected by the following phage: H, H-1, H-2, H-3, H-4, Lucas 110, Lucas 303, Lucas 404, YerA3, YerA7, YerA20, YerA41, 3/M64-76, 5/G394-76, 6/C753-76, 8/C239-76, 9/F18167, 1701, 1710, PST, 1/F2852-76, D'Hérelle, EV, H, Kotljarova, PTB, R, Y, YerA41, φYerO3-12, 3, 4/C1324-76, 7/F783-76, 903, 1/M6176 and Yer2AT.

In particular, bacteria species (and corresponding, host-specific bacteriophages) include Aeromonas hydrophila (PM2), Bacillus anthracis (Gamma), Bacillus subtilus (SPP1), Bordetella pertussis (See N. A. Pereversev et al. 1981 Zh Mikrobiool 5:54-57), Borrelia burgdorferi (φBB-1, see Eggers et al. 2001 J Bacteriol 183:4771-4778), Brucella abortus (TB; 212; 371), Campylobacter jejuni (φ4, φC), Clostridium perfringes (φ3626), Enterococcus faecalis (φFC1), Enterococcus faecium (ENB6), Escherichia coli (P1; T1; T3, T4, T5; T7, KH1, φV10; lambda; φ20; mu), Klebsiella pneumoniae (60; 92), Listeria monocytogenes (A511, A118; 243; H387; 2389; 2671; 2685; 4211), Mycobacterium leprae (mycobacteriophage, L5), Mycobacterium tuberculosis (LG; DSGA), Pseudomonas aeruginosa (E79, G101; B3; pp. 7), Salmonella anatum (E5), Salmonella bovismorbificans (98), Salmonella choleraesuis (102), Salmonella enteritidis (L; P22; 102; FO; IRA; φ8), Salmonella Newington (E34), Salmonella schottmulleri (31; 102; F0; 14), Salmonella typhi (163; 175; ViI; ViVI; 8; 23; 25; 46; 175; F0), Serratia marcescens (S24VA), 517Shigella dysenteriae (φ80; P2; 2; 37), Shigella flexneri (Sf6), Staphylococcus aureus (K; P1; P14; UC18; 15; 17; 29; 42D; 47; 52; 53; 79; 80; 81; 83A; 92; Twort, φ11), Streptococcus pneumoniae (Dp-1; Cp-1; HB-3; EJ-1; MM1; VO1), Streptococcus pyogenes (φX240; 1A; 1B; T12; 12/12; 113; 120; 124; P58; H4489a), Vibrio cholerae (138; 145; 149; 163), and Yersinia pestis (A1122; R; Y; P1).

### Bacteriophage Packaging Sites

An important aspect of the present invention is the use of a bacteriophage packaging site (the specific DNA sequence on the phage genome that is required for genome packaging into the virion). The plasmid is engineered to contain a phage packaging site so that plasmid is packaged into the transducing particles. Host-specific bacteriophages (and their packaging sites) include but are not limited to SPP1 (SPP1 pac site), P1 (P1 pac site), T1 (T1 pac site), T7 (T7 concatamer junction), lambda (cos site), mu (mu pac site), P22 (P22 pac site), φ8 (φ8 pac site), Sf6 (Sf6 pac site), 149 (149 pac site), and A1122 (A1122-concatamer junction). For most bacteriophages, the packaging site is termed the pac site. In some cases, the packaging site is referred to as a concatamer junction (e.g. T7 concatamer junction). In every case, the packaging site is substantially in isolation from sequences naturally occurring adjacent thereto in the bacteriophage genome.

For some bacteriophages, the packaging site may be unknown. In these cases, pac sites can be determined by taking advantage of the property that plasmids containing a functional bacteriophage pac site are packaged. For example, the DNA sequences necessary for packaging of bacteriophage λ were determined by incorporating small restriction fragments of the λ phage genomic DNA into a plasmid (Hohn, B 1983 PNAS USA 80:7456-7460). Following introduction into an in vivo packaging strain, the efficiency of packaging/transduction was quantitatively assessed. Using a similar strategy, the pac sites for a number of bacteriophages have been determined: λ(Miwa, T 1982 Gene 20:267-279); Mu (Croenen, M A and van de Putte, P 1985 Virology 144:520-522); filamentous bacteriophages including f1, fd, M13, and Ike (Russel, M and Model, P 1989 J Virol 1989 63:3284-3295); P22 (Petri, J B and Schmieger, H 1990 Gene 88:47-55; Wu, H et al. 2002 Molec Microbiol 45:1631-1646); T7 (Chung, Y B and Hinkle, D C 1990 J Mol Biol 216:927-938), and T3 (Hashimoto, C and Fujisawa, H 1992 Virology 187:788-795).

Embodiments of the invention include bacteriophage packaging sequences and functional fragments thereof. These nucleic acid embodiments can be for example, at least 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 250, 300, 350, 400, 450, 500, 550, 600, 650, 700, 750, 800, 850, and 900 nucleotides in length so long as the nucleotide fragment can mediate packaging of plasmid DNA into bacteriophage capsids (as judged by its ability to mediate packaging and thereby produce functional transducing particles). The nucleic acids that comprise the bacteriophage packaging sites or fragments thereof are incorporated into the plasmids of the present invention.

### Reporter Genes

Reporter gene technology is widely used to monitor cellular gene expression (Naylor, L H 1999 Biochem Pharm 58:749-757). Commonly used reporter genes include chloramphenicol acetyltransferase (CAT), 13-galactosidase, luciferase, alkaline phosphatase, and green fluorescent protein (GFP). In general, reporter genes have the advantage of low background activity and sensitive signal detection following gene expression. For example, the development of luciferase and GFP as non-invasive markers of gene expression, combined with ease of detection using sensitive charge-coupled device (CCD) imaging cameras and fluorescence microscopy, has allowed for temporal and spatial information about gene expression even at the single cell level.

A review of luciferase genes and their use as reporter genes provides a list of known luciferase genes, cDNAs, proteins, and corresponding Gen Bank Accession numbers (Greer, L F and Szalay, A A 2002 Luminescence 17:43-74, see Table 1, pp. 45-46). Greer and Szalay 2002 also summarize a large number of constructs and vectors that are useful for imaging (see Table 2, pp 48-52). These vectors are suitable for expression in Staphylococcus aureus, E. coli and other bacteria. Among the known luciferases are the prokaryotic luciferases (Lux), eukaryotic luciferases (Luc, Ruc and their regulatory proteins) both of which are commonly used in imaging of luciferase expression in living cells and organisms.

The demonstration that GFP from jellyfish Aequorea victoria required no jellyfish-specific cofactors and could be expressed as a fluorescent protein in heterologous hosts including both prokaryotes and eukaryotes sparked the development of GFP as one of the most common reporters in use today (Southward, C M and Surette M G 2002 Molec Microbiol 45:1191-1196). In addition, spectral variants with blue, cyan and yellowish-green emissions have been generated from Aequorea GFP. GFP-like proteins have been expanded to include about 30 significantly different members. A list of GFP-lilce proteins and corresponding Genbank accession numbers can be found in Miyawaki, A 2002 Cell Struct and Funct 27:343-347 (see Table I, p. 344).

A β-galactosidase reporter gene can also be used to detect gene expression in bacteria (Miller J. H. ed 1972, in Experiments in Molecular Genetics, Cold Spring Harbor Laboratory, Cold Spring Harbor, N.Y.; Sambrook, J et al. 1989 in Molecular Cloning, Cold Spring Harbor Laboratory, Cold Spring Harbor, N.Y.). β-galactosidase activity expressed by bacterial colonies is detected by blue coloration on medium containing X-Gal (5-bromo-4-chloro-3-indolyl-β-D-galactopyranoside).

Chloramphenicol acetyltransferase (CAT) is also suitable for use as a reporter gene in bacteria. CAT is encoded by a bacterial drug-resistance gene (Kondo, E and Mitsuhashi, S1964 J Bacteriol 88:1266-1276). It inactivates chloramphenicol by acetylating the drug at one or both of its two hydroxyl groups. In a typical CAT assay, cell extracts are incubated in a reaction mix containing ¹⁴C- or ³H-labeled chloramphenicol and n-Butyryl Coenzyme A. CAT transfers the n-butyryl moiety of the cofactor to chloramphenicol. The reaction products are extracted with xylene and the n-butyryl chloramphenicol partitions mainly into the xylene phase, while unmodified chloramphenicol remains predominantly in the aqueous phase. Radiolabeled chloramphenicol that partitions into the xylene phase is measured using a scintillation counter.

Bacterial alkaline phosphatase encoded by phoA of Escherichia coli is enzymatically active only when it has been transported across the cellular membrane into the periplasmic space (Gibson, C M and Caparon, M G 2002 Appl and Env Microbiol 68:928-932). This property has been exploited to engineer PhoA protein as a molecular sensor of subcellular location (for a review, see Manoil, C et al. 1990 J Bacteriol 172:515-518). Another bacterial alkaline phosphatase (PhoZ) derived from the gram-positive bacterium Enterococcus faecalis (Lee, M H 1999 J Bacteriol 181:5790-5799) has been developed as a reporter that is highly active in gram-positive bacteria (Granok A B 2000 J Bacteriol 182:1529-1540; Lee M H 1999 J Bacteriol 181:5790-5799). The alkaline phosphatase activity of PhoZ, like that of PhoA, is dependent on its export from the cytoplasm. In an alkaline phosphatase assay, alkaline phosphatase hydrolyzes substrates such as 4-nitrophenyl phosphate (4NPP) to yield a chromogen (e.g. 4-nitrophenol, 4NP).

Reporter genes allow for simpler manipulation procedures (e.g. reduced purification or cell lysis), they are adaptable to large-scale, high throughput screening measurements, and they are compatible with bacteria systems. Reporter genes can be either naturally occurring genes or those produced by genetic manipulation, such as recombinant DNA technology or mutagenesis. Reporter genes are nucleic acid segments that contain a coding region and any associated expression sequences such as a promoter, a translation initiation sequence, and regulatory sequences.

### Bacteria-Specific Promoters

The reporter gene is linked to a promoter sequence that controls and directs synthesis of RNA. It will be appreciated by those of ordinary skill in the art that a promoter sequence may be selected from a large number of bacterial genes expressed by various bacterial species. The choice of promoter is made based on the target bacteria to be detected. For a review of strategies for achieving high-level expression of genes in E. coli, see Makrides, S C 1996 Microbiol Rev 60:512-538. An exemplary promoter sequence effective in E. coli is the T7 promoter, but any promoter or enhancer that is functional in prokaryotic cells may be used. Useful promoters include, but are not limited to, lac promoter (E. coli), trp promoter (E. coli), araBAD promoter (E. coli), lac hybrid promoter, (E. coli), trc hybrid prormoter (E. coli), PL (X), SP6, and T7.

A promoter sequence used in the present invention is selected on the basis of its ability to achieve a detectable level of expression in the target pathogenic bacteria. In a preferred embodiment, the reporter gene is preferably coupled to a promoter obtained from the pathogenic bacterial host to be detected. A constitutive promoter will express the reporter at a constant rate regardless of physiological demand or the concentration of a substrate. Alternatively, it may be advantageous to use an inducible promoter to control the timing of reporter gene expression. For inducible promoters such as the lac and trp operons, expression is normally repressed and can be induced at a desired time. In the absence of lactose, the lac promoter is repressed by lac repressor protein. Induction can be achieved by the addition of lactose or IPTG, preventing the binding of repressor to the lac operator. Similarly, the lip promoter is negatively regulated by a tryptophan-repressor complex that binds to the trp operator. For the trp operon, gene expression can be induced by removing tryptophan or by adding β-indoleacrylic acid.

### Bacteria-Specific Origins of Replication

Origins of replication used in the plasmids of this invention may be moderate copy number, such as colE1 ori from pBR322 (15-20 copies per cell) or the R6K plasmid (15-20 copies per cell) or they may be high copy number, e.g. pUC oris (500-700 copies per cell), pGEM oris (300-400 copies per cell), pTZ oris (>1000 copies per cell) or pbluescript oris (300-500 copies per cell). The origins of replication may be functional in E. coli or in any other prokaryotic species such as Bacillis anthracis or Yershinia pestis.

Plasmid replication depends on host enzymes and on plasmid encoded and plasmid-controlled cis and trans determinants. For example, some plasmids have determinants that are recognized in almost all gram negative bacteria and act correctly in each host during replication initiation and regulation. Other plasmids possess this ability only in some bacteria (Kues, U and Stahl, U 1989 Microbiol Rev 53:491-516). Plasmids are replicated by three general mechanisms, namely theta type, strand displacement, and rolling circle (reviewed by Del Solar et al. 1998 Microbio and Molec Biol Rev 62:434-464).

For replication by the theta type mechanism, the origin of replication can be defined as (i) the minimal cis-acting region that can support autonomous replication of the plasmid, (ii) the region where DNA strands are melted to initiate the replication process, or (iii) the base(s) at which leading-strand synthesis starts. Replication origins contain sites that are required for interactions of plasmid and/or host encoded proteins. Plasmids undergoing theta type replication also include pPS10, RK2 (containing oriV), RP4, R6K (containing oriy), ColE1 and CoIE2. ColE1 is the prototype of a class of small multicopy plasmids that replicate by a theta-type mechanism. The origin of C61E1 replication spans a region of about 1 kb (Del Solar et al. 1998).

Examples of plasmids replicating by the strand displacement mechanism are the promiscuous plasmids of the IncQ family, whose prototype is RSF1010. Members of this family require three plasmid-encoded proteins for initiation of DNA replication. These proteins promote initiation at a complex origin region, and replication proceeds in either direction by a strand displacement mechanism. The origin of replication has been defined as the minimal region able to support bidirectional replication when the RSF110 replication proteins (RepA, RepB, and RepC) are supplied in trans by a second plasmid. The minimal ori region includes three identical 20-bp iterons plus a 174 bp region that contains a GC-rich stretch (28 bp) and an AT-rich segment (31 bp) (Del Solar et al. 1998).

Replication by the rolling circle (RC) mechanism is unidirectional, and is considered to be an asymmetric process because synthesis of the leading strand and synthesis of the lagging strand are uncoupled. Studies on the molecular mechanisms underlying RC replication have been done mainly with the staphylococcal plasmids pT181, pC221, pUB110, pC194, and with the streptococcal plasmid pMV158 and its Amob derivative pLS1. Other plasmids or phage that undergo R^{C} replication include but are not limited to pS194, fd, φX174, pE194 and pFX2 (Del Solar et al. 1998).

Prokaryotes have a circular molecule of chromosomal DNA, typically with a single origin of replication. For example, the chromosomal origin of replication of E. coli and other bacteria is termed oniC. The present invention envisions the use of origins of replication known in the art that have been identified from species-specific plasmid DNAs (e.g. ColE1, R1, pT181, and the like discussed herein above), from bacteriophages (e.g. φX174 and M13) and from bacterial chromosomal origins of replication (e.g. oriC).

### Antibiotic Resistance Genes

The plasmid DNA of the transducing particles of the invention will optionally have an antibiotic resistance gene to facilitate molecular biology cloning of the plasmid and to allow for selection of bacteria transformed by plasmid. Antibiotic resistance genes are well known in the art and include but are not limited to ampicillin resistance (Amp^{r}), chworamphenicol resistance (Cm^{r}), tetracycline resistance (Tet^{r}), kanamycin resistance (Kan^{r}), hygromycin resistance (hyg or hph genes), and zeomycin resistance (Zeo^{r}).

### Methods of Making Transducing Particles

The transducing particles of the present invention are obtained by modifying a naturally-occurring bacteriophage to carry a gene capable of transforming the target bacteria to an easily recognizable phenotype, referred to hereinafter as the reporter gene. The transducing particle must be capable of specifically introducing the reporter gene into the target bacterial host in such a way that the bacterial host can express the gene function in a detectable manner. A large number of bacteriophages exist and may be selected for modification based on the desired host range and the ability of the bacteriophage to carry and transduce the gene of interest. In particular, the bacteriophage must be large enough to accommodate the reporter gene, the associated promoter region, the phage packaging site and any other DNA regions which may be included. Modified bacteriophages of the present invention will usually retain the normal host range specificity of the unmodified bacteriophage, although some alteration in specificity would be acceptable so long as it does not affect the ability to identify the selected target bacteria.

The bacteriophages to be modified may be temperate or virulent, preferably being temperate. Modification of the bacteriophage results in a defective transducing particle that is capable of introducing the reporter gene into a target bacterial host, but which is incapable of achieving lytic or lysogenic infection. The reporter gene is part of a plasrid or other self-replicating episomal unit which will be sustained and expressed in the infected host.

Transduction of the reporter gene may take place via transient expression (i.e., expression from a reporter gene which is not stably inherited by the cell) of the reporter gene. In such case, the DNA transduced by the bacteriophage may not survive intact through the entire test period. However, transcription of the reporter gene transduced by the phage will be sufficiently efficient before the DNA is degraded to ensure that the bacteria has assembled a functional reporter gene by the end of the test period. The bacteria can thus be detected by the assay of the invention even if the bacteria degrades the phage DNA.

Bacteriophages useful in the present invention may be obtained from microbiological repositories, such as the American Type Culture Collection, P.O. Box 1549, Manassas, Va., 20108, USA. Virulent bacteriophages are available as bacteria-free lysates, while lysogenic bacteriophages are generally available as infected host cells.

Wild-type bacteriophage obtained from any source may be modified by conventional recombinant DNA techniques in order to introduce a desired reporter gene capable of producing the detectable phenotype of interest. Prior to introduction, the reporter gene of interest will be combined with a promoter region on a suitable gene cassette. The gene cassette may be constructed by conventional recombinant DNA techniques in a suitable host, such as E. coli. Both the reporter gene and the promoter region should be chosen to function in the target host, and the cassette may optionally include a second reporter gene, such as antibiotic resistance, heavy metal resistance, or the like, to facilitate in vitro manipulation.

The reporter gene (or genes, if not a single gene system) should be capable of expressing a screenable phenotype in the target bacterial host. As used hereinafter, the phrase screenable phenotype is intended to mean a characteristic or trait which allows cells that express the phenotype to be distinguished from other cells which do not express the phenotype, even when all cells are growing and reproducing normally in a mixed culture. That is, detection of the characteristic or trait may be carried out while the infected target cells are present in mixed population of viable, usually proliferating non-target bacteria which do not express the phenotype. Preferably, the screenable phenotype will comprise a visually observable trait, i.e., one that can be directly or indirectly observed in a mixed population of target and non-target cells. The phenotype will usually not be selectable, i.e., one which provides for survival or preferential growth under particular conditions (positive selection) or which provides for growth inhibition or killing under particular conditions. The method of the present invention does not require that target bacteria present in the sample be isolated from or enriched relative to non-target bacteria which may be present in the sample because the trait will be observable when target bacteria comprise only a portion of the viable bacteria present.

The reporter gene can encode the screenable phenotype by itself or may be part of a multiple gene system encoding the phenotype, where other genes are present in or separately introduced to the host to be detected. For example, the transducing particle may carry the lacZα gene which requires a complementary lacZβ gene or lacZΔM15 deletion in the host for expression.

Suitable screenable phenotypes include bioluminescence, fluorescence, enzyme-catalyzed color production (e.g., using the enzyme alkaline phosphatase), and the like. Each of these phenotypes may be observed by conventional visualization techniques which provide the chemical reagents necessary to complete a signal producing reaction. Preferred is the use of luciferase activity, although the present invention may be achieved with other detectable phenotypes.

For the bacteriophage, it is possible to package the plasmid or the reporter gene cassette by attachment of the bacteriophage packaging site in a DNA construct with the plasmid or cassette. The packaging site may be obtained from the bacteriophage genome and cloned into the plasmid carrying the reporter gene, promoter region, and optional second reporter. The plasmid may then be transferred to a suitable bacterial host. The bacterial host will then produce transducing particles having the plasmid and/or marker gene cassette packaged within a bacteriophage coat capable of inserting the plasmid DNA into bacteria of its host range. The plasmid is transposed into the desired bacteriophage by simultaneous infection of a suitable host with both the plasmid and the bacteriophage. The host cells are incubated and the transducing particles are collected. A fraction of the phage will be carrying the plasmid. The transducing particles can be separated from the phage by conventional techniques.

In the invention, the host-specific bacteriophage packaging sites of the invention are substantially in isolation from sequences naturally occurring adjacent thereto in the bacteriophage genome. As used herein, the term “substantially in isolation” with respect to bacteriophage packaging sites, means they that are not in their natural environment. That is, the packaging sites are not in a full-length, bacteriophage genomic nucleic acid sequence found in nature. The packaging sites may be isolated from the full length bacteriophage genomic sequence via experimental techniques, such as use of restriction endonuclease enzymes and cloning or amplification by the polymerase chain reaction. The packaging sites also may be produced synthetically.

A bacteriophage packaging site of the present invention is a nucleic acid fragment devoid, in whole or part, of sequences normally associated with it in nature; or a sequence, as it exists in nature, but having heterologous sequences in association therewith. It is a fragment disassociated from the bacteriophage genome.

As used herein, the phrase “functional equivalents” in the context of bacteriophage packaging sites means packaging sites that function the same, qualitatively, as the wild type bacteriophage packaging sites. Thus, if an isolated bacteriophage packaging site directs packaging of DNA, a DNA fragment would be a functional equivalent if it also directs packaging of DNA in the same manner. Quantitative equivalence is not needed for a fragment to be a functional equivalent according to this invention. Thus bacteriophage packaging sites that have nucleotide substitutions, deletions and/or additions can be functional equivalents of an isolated bacteriophage packaging site.

### Methods of Using Transducing Particles

Transducing particles prepared as described above are used to detect target bacteria in biological samples as follows. In some instances it will be possible to infect a biological sample and observe the alteration and phenotype directly, although in other cases it may be preferred to first prepare a mass culture of the bacteria present in the sample. Methods for obtaining samples and (if necessary) preparing mass culture will vary depending on the nature of the biological sample, and suitable techniques for preparing various sample types are described in detail in standard microbiology and bacteriology texts such as Bergey's Manual of Determinative Bacteriology (8th ed.), Buchanan and Gibbons (eds.) Williams & Wilkens Co., Baltimore (1974); Manual of Methods for General Bacteriology, Gerhardt et al. (eds.), Am. Soc. Microbiology, Wash. (1981); and Manual of Clinical Microbiology (8th ed.), Patrick, R et al. (eds.), Am. Soc. Microbiology, Washington (2003).

Once the biological sample has been prepared (with or without growth of a mass culture), it will typically be exposed to transducing particles under conditions which promote binding of the particles to the bacteria and injection of the genetic material, typically at a temperature which supports rapid growth of the bacteria (e.g., 35° C. to 40° C.) without agitation for a time sufficient to allow infection (e.g., 15 minutes to 120 minutes). Following infection, the cells are incubated to allow expression of the reporter gene and reporter gene expression is detected as described above.

The method of the present invention will be used most frequently to screen for a specific type of bacteria (as determined by the host range of the transducing particle) in a mixed population of bacteria derived from a biological sample as described above. The mixed bacterial populations need not be selected prior to screening. Preparation of the sample prior to screening will generally not provide a homogeneous bacterial population, although it is possible to combine the screen of the present application with nutritional selection as described below.

In contrast to conventional phage transduction techniques intended to produce homogeneous colonies of transduced bacterial cells, the method of the present invention does not require that the transduced bacteria be isolated in any way. Instead, the screenable phenotype, e.g., a visually observable trait, conferred by the reporter gene can be detected in a non-selected portion of the biological sample where viable, usually proliferating, non-target bacteria will be present. The screening can occur without selection since there is no need to isolate the transduced bacteria.

As described above, the assay of the present invention is useful for screening biological samples to determine whether bacteria present in the host range of the transducing particles are present. The present invention is also useful for typing bacterial species and strains in a manner similar to conventional phage typing which instead relies on much slower plaque assays for determining phage infection.

For typing according to the present invention, a panel of transforming particles having differing, usually overlapping, host ranges are employed. The species and strain of the target bacteria may then be determined based on the pattern of reactivity with the various transforming particles. Often, such tests may be run on a single carrier, where the different transforming particles are spotted in a fixed geometry or matrix on the carrier surface. The pattern of reactivity may then be rapidly observed. In contrast to the previously-described screening methods, these typing methods will be useful in characterizing homogeneous bacterial cultures (i.e., contained on a single species or strain) as well as typing target bacteria in mixed populations.

The present invention may be combined with nutritional screening in order to further characterize the bacteria being investigated. By providing a selective medium during either the mass culture or the plating culture, the range of bacteria which can remain viable may be limited. As the phenotypic assay of the present invention can only detect viable cells, absence of a detectable phenotype limits the type of bacteria which may be present. By properly combining the host range of the transducing particles and the viability range of the selective medium, the method of the present invention can be made very specific for the type of bacteria being determined.

A second approach for increasing the ability of the present invention to specifically identify bacterial hosts involves the use of immunoadsorption. Immobilized antibodies, including antisera or monoclonal antibodies, are utilized to specifically capture bacterial cells based on the identity of their cell surface epitopes. The bacteria may then be further detected using the transducing particles of the present invention, as described above. Suitable materials and methods for the immunoadsorption of particular bacterial species and strains on solid phase surfaces are described in Enterobacterial Surface Antigens: Methods for Molecular Characterization, Korhonen et al. (eds.), Elsevier Science Publishers, Amsterdam (1986).

The present invention can be particularly useful in patient diagnosis as it allows the determination of bacterial sensitivity to antibiotics and other bactericides. By performing a short incubation of the bacteria with an antibiotic or bactericide to be screened prior to exposure to the transducing particles of the present invention, the metabolic activities of the cells will be halted and the alteration of the phenotype prevented. Such testing will be useful after the presence of the bacteria is initially confirmed using the transforming particles as described above. Antibiotics and bactericides which are determined to be lethal to the bacterial infection may then be employed for treatment of the patient. Such rapid and early detection of useful antibiotics and bactericides can be invaluable in treating severe bacterial infections.

Similarly, the present invention can be useful in detecting the presence of antibiotics, e.g., antibiotic residues in animal products. In this approach, an extract of the material to be analyzed is added to a culture of a bacterial strain sensitive to the antibiotic in question, and the mixture is incubated for a period predetermined to be sufficient to kill the strain if a given amount of antibiotic is present. At this point, transducing particles of the invention specific to the strain are added, and the assay of the invention is performed. If the given amount of antibiotic is present, the cells of the bacterial strain will be dead and the read-out will be negative (i.e., lack of luminescence in a luciferase assay). If the given amount of antibiotic is not present, cells of the bacterial strain will survive and the read-out will be positive (i.e., luminescence in a luciferase assay).

In a specific embodiment, a means is provided for assaying bacteria which have been previously rendered susceptible to transducing particles of the invention on a phage-specific basis. That is, in a first step, the target bacteria are modified, e.g., by transformation, so that they contain or express a cell-specific receptor for the bacteriophage of interest. In a second step, the modified (or tagged) bacteria are introduced into, or mixed into, a sample environment in which they are to be followed. The sample environment can be any setting where bacteria exist, including outdoors (e.g., soil, air or water); on living hosts (e.g., plants, animals, insects); on equipment (e.g., manufacturing, processing or packaging equipment); and in clinical samples. The bacteriophage assay of the invention (as described previously) can then be carried out, using bacteriophage specific for the introduced receptor, and the presence of the tagged bacteria can be monitored or quantified.

An advantage of this embodiment is that it provides a means to follow or track bacteria to be released into a sample environment wlich already contains the same type of bacteria (or closely similar bacteria) or which may be subject to introduction of the same type of bacteria (or closely similar bacteria) from a separate source. The bacteria being tracked can be distinguished from the other bacteria (i.e., bacteria which are essentially the same) by virtue of the presence of the cell-specific receptor which has been introduced into the bacteria being tracked. There is thus provided the opportunity of assaying for the presence of released bacteria in the presence of otherwise identical (but for the receptor component) bacteria, without cross reactivity (background).

### Example 1

### Reporter Plasmid Phage Packaging System for Detection of Bacteria

Bacteriophage are typically highly specific for a given species or strain of bacteria. This specificity can be exploited for the detection of a given species/strain of bacteria from an environmental or medical sample that may contain many different bacteria types. One strategy is to engineer reporter genes such as luciferase or green fluorescent protein into the phage genome such that the reporter gene is expressed and can be detected upon infection of the target bacteria. This concept is well documented. Nevertheless, this system has limitations, particularly when the only phages available for a given bacteria species are lytic and can rapidly kill the target cell before there is significant expression of the reporter gene.

This new invention overcomes the above-mentioned limitations but still makes use of the specificity of bacteriophage to its host. Briefly, a plasmid which is capable of stable replication in a given host is engineered to contain a reporter gene as well as the phage packaging site (the specific DNA sequence on the phage genome that is required for genome packaging into the virion). This construct is transformed into a bacterial host, and then infected with the specific bacteriophage. Because the packaging site is on the plasrid, a percentage of the progeny phage particles will have reporter plasmids packaged into the heads instead of the phage genome. When these particles are contacted with target bacteria, the phage will inject the plasmid into the cell that can then be detected by expression of the reporter gene.

We have developed a model system using bacteriophage T7 and the bacteria E. coli and contemplate a similar system for detecting Y. Pestis with bacteriophage A1122.

### Construction of the Phage Reporter Packaging Plasmid

Plasmid pGFPuv (Clontech) was digested with Eagl and EcoRT (these sites are in the 3′ multiple cloning site of the plasmid). pGFPuv encodes the green fluorescent protein driven from a bacterial promoter and an ampicillin resistance gene. A DNA fragment from plasmid pRDI (from Chung and AEnkle 1990 J Mol Biol 216:911-926) was amplified by PCR and cloned into the Eagl/EcoRI sites of pGFPuv. This amplified fragment contains nucleotides 1-439 and 38981-39937 of bacteriophage T7 (Accession: NC_{—}001604). This DNA sequence contains the information for packaging the T7 DNA genome into the phage capsid. This construct was named pPAE. pPAE was transformed into E. coli strain BL21 (common lab strain). These transformants were infected with T7. Because the pPAE contains the packaging sequences, some of the progeny phage particles in the lysate contained pPAE instead of a phage genome. We call these particles “transducing particles”. When the lysate was contacted with a fresh culture of BL21, the transducing particles injected the plasmid into the cells they infected. These cells were then plated on ampicillin plates. Colonies that grew contained pPAE and expressed GFP which could be detected by, green color when exposed to UV light. The plasmid construct of pPAE was confirmed by PCR.

### Assay Using BL21 and T7 as a Model.

1. Construct a lysate containing transducing particles from pPAE.
2. Put this lysate along with some growth media into a tube.
3. Add a sample that you suspect may contain BL21.
4. Incubate.
5. Measure fluorescence. Any fluorescence above background means that some BL21 was present, the particles injected the plasmid, and GFP got expressed. There is no other way that GFP could be expressed without the target bacteria.

### Commercial Applications

Detection of specific bacteria in an environmental or medical sample. Commercial uses: biodefense, food industry for contaminating bacteria, medical diagnosis for infectious disease. General method for packaging any DNA into a phage capsid, could be used for gene therapy etc.

While the present invention has been described in some detail and form for purposes of clarity and understanding, one skilled in the art will appreciate that various changes in form and detail can be made without departing from the true scope of the invention. All figures, tables, and appendices, as well as patents, applications, and publications, referred to above, are hereby incorporated by reference.

What is claimed is:

1. A composition of matter comprising a transducing particle that comprises a bacteriophage coat and a DNA core that comprises plasmid DNA comprising
a) a host-specific bacteriophage packaging site wherein the packaging site is substantially in isolation from sequences naturally occurring adjacent thereto in the bacteriophage genome,
b) a reporter gene,
c) a bacteria-specific promoter operably linked to said reporter gene,
d) a bacteria-specific origin of replication, and optionally
e) an antibiotic resistance gene.

2. A method for producing transducing particles that comprise a bacteriophage coat and a DNA core that comprises plasmid DNA, said method comprising:
a) introducing plasmid DNA into a bacterial host, wherein the plasmid DNA comprises
i) a host-specific bacteriophage packaging site wherein the packaging site is substantially in isolation firom sequences naturally occurring adjacent thereto in the bacteriophage genome,
ii) a reporter gene,
iii) a bacteria-specific promoter operably linked to said reporter gene,
iv) a bacteria-specific origin of replication, and optionally
v) an antibiotic resistance gene,
b) infecting the bacterial host with a host-specific bacteriophage, and
c) collecting progeny phage transducing particles that comprise said plasmid DNA.

3. A method of detecting target bacteria present in a biological sample, said method comprising:
a) exposing a biological sample to a transducing particle, wherein said transducing particle comprises a bacteriophage coat and a DNA core that comprises plasmid DNA, said plasmid DNA comprising:
i) a host-specific bacteriophage packaging site wherein the packaging site is substantially in isolation from sequences naturally occurring adjacent thereto in the bacteriophage genome,
ii) a reporter gene,
iii) a bacteria-specific promoter operably linked to said reporter gene,
iv) a bacteria-specific origin of replication, and optionally
v) an antibiotic resistance gene,
b) incubating said bacterial sample under conditions to allow delivery of said plasmid DNA into the bacteria, and
c) monitoring expression of said reporter gene comprised within said plasmid DNA, thereby detecting target bacteria present in the biological sample.
